(19) United States
(12) Patent Application Publication
(10) Pub. No.: US 2026/0356651 A1
(43) Pub. Date: Nov. 12, 2026

(54) DISPENSING UNIT FOR DISPENSING MIXED FLUIDS

(71) Applicant: Aquablu Holding B.V., Amsterdam (NL)

(72) Inventors: Max Daniel Boo HOEBOER, Amsterdam (NL); Marnix Jacob Paul STOKVIS, Amsterdam (NL); Marc Leopold Robert VAN ZUYLEN VAN NIJEVELT, Amsterdam (NL); Mees BRINKHUIS, Amsterdam (NL)

(73) Assignee: Aquablu Holding B.V., Amsterdam (NL)

(21) Appl. No.: 19/125,049

(22) PCT Filed: Oct. 23, 2023
(86) PCT No.: PCT/NL2023/050558

§ 371 Date: Apr. 28, 2025

(30) Foreign Application Priority Data
Oct. 28, 2022 (NL) 2033419

(51) Int. Cl. B67D 1/00; A61L 2/104; B01F 23/40; B01F 23/45; B01F 25/23; B01F 25/25; B01F 35/11; B01F 101/14; B08B 3/04; B67D 1/07; B67D 1/08

(52) U.S. Cl. CPC B67D 1/0016; A61L 2/104; B01F 23/45; B01F 23/483; B01F 25/23; B01F 25/25; B01F 35/11; B08B 3/04; B67D 1/0046; B67D 1/07; B67D 1/0882; A61L 2202/11; B01F 2101/14; B67D 2001/075

(57) ABSTRACT

Dispensing unit, for dispensing mixed fluids, comprising at least one mixing chamber, for mixing a supply fluid and an additive into a fluid mixture, at least one fluid supply connection, for providing at least one supply fluid to the mixing chamber, at least one additive supply, for providing at least one additive to the mixing chamber, preferably directly or indirectly into the supply fluid, at least one fluid outlet, for dispensing a fluid mixture from the mixing chamber, at least one disinfection element.

The present invention relates to a dispensing unit for dispensing mixed fluids, a beverage dispensing system comprising such dispensing unit and a method for dispensing flavoured water, comprising a dispensing unit.

Beverage dispensing machines are wide-spread, in the food industry but also in domestic use such as households. Typically such machines are able to dispense various drinks or beverages on command from multiple outlets. There are also machines available which dispense beverages with flavour additives.

US2022/0119239 for example relates to flavor and additive delivery systems and methods for beverage dispensers. An example beverage dispenser includes flavor containers configured to contain flavor mixtures, a manifold configured to blend water with flavor mixtures, a flowrate sensor configured to detect a current flowrate of the water flowing to the manifold, memory configured to store instructions related to dispensing the water and the flavor mixtures, and a processor. The processor is configured to receive a request for a selected beverage, identify a ratio between the water and one or more of the flavor mixtures for the selected beverage, transmit a water control signal to cause the water to flow at a requested water flowrate, and transmit one or more first flavor control signals to cause one or more of the flavor mixtures to flow from the flavor containers based on the ratio.

Although such systems appear to operate, a problem is that the machines contaminate themselves with flavour additives. The additives often contain sugar or a similar compound, which provides a fruitful base for bacteria to grow if not dealt with on a regular basis. The present invention thus has the goal to provide an improved dispensing system wherein contamination of the system with additives is reduced or eliminated.

Therefore, the present invention provides a dispensing unit, for dispensing mixed fluids, comprising:
At least one mixing chamber, for mixing a supply fluid and an additive into a fluid mixture;
At least one fluid supply connection, for providing at least one supply fluid to the mixing chamber,
At least one additive supply, for providing at least one additive to the mixing chamber, preferably directly or indirectly into the supply fluid,
At least one fluid outlet, for dispensing a fluid mixture from the mixing chamber,

Preferably, at least one disinfection element, preferably a UV disinfection element, for disinfecting at least a portion of the mixing chamber and/or the at least one additive supply, in particular at least during use and/or at least not during use.

The dispensing unit according to the present invention is in particular advantageous for dispensing a flavoured water beverage to the desires of a user. The supply fluid can be any type of water (tap, bottled, carbonated, filtered or a combination thereof) and as an additive flavours can be used. Other types of additives are also conceivable, such as colorants, vitamins, minerals. Preferably the additives are also in a liquid form, but powders are also conceivable. In case of powder or solid additives it is particularly preferred that the powder and/or solid is soluble, more in particular water soluble. The dispensing unit preferably allows for dispensing a good stream of fluid mixture from the fluid outlet, This may be understood as a stream which does not splash excessively in all directions, but is directed in a controlled manner towards a fluid retaining element, such as a holder below the dispensing opening.

The dispensing unit may further comprise a housing. The at least one mixing chamber, at least one additive supply, at least one fluid outlet and at least one disinfection element may be mounted to or form an integral part of the housing, to protect and shield the components from the environment. The at least one fluid supply connection may be directly or indirectly connected to the housing. Preferably the mixing chamber is part of a mixing module, which can be replaceable connected with the housing. This way the mixing chamber can be cleaned individually or can be replaced with other components such as dedicated cleaning units or cleaning chambers. Alternatively, the mixing chamber can form an integral part of the dispensing unit. Preferably, the dispensing unit comprises a central axis. The mixing chamber can optionally have the same central axis as the central axis of the dispensing unit itself.

Typically the mixing chamber is defined by a top, a bottom and walls, or a circumferential wall, which extend from the top to the bottom. The bottom may also taper into a cone-like shape. The mixing chamber is preferably positioned below and downstream of the fluid supply connection. Preferably, the mixing chamber comprises an open top opening for receiving the supply fluid. The bottom of the mixing chamber is preferably open, allowing the fluid mixture to exit the mixing chamber and/or dispensing unit. It is imaginable that the bottom opening of the mixing chamber forms the at least one fluid outlet, although the outlet may also be provided further downstream. The mixing chamber may have a direct or indirect connection with the fluid outlet. Yet, the mixing chamber may also define the fluid outlet. The mixing chamber can be an integrated mixing zone or section of the dispensing unit. Preferably the mixing chamber comprises a substantially circular outer wall for guiding the supply fluid through the mixing chamber towards the fluid outlet. More preferably, wherein said outer wall forms a funnel shape, narrowing towards the fluid outlet. The fluid mixture can be defined as a fluid stream with uniformly distributed and/or mixed additive. Preferably the mixing chamber is positioned in between the fluid supply connection and the fluid outlet, wherein the mixing chamber is positioned upstream and above the fluid outlet. In particular the mixing chamber is positioned vertically straight above the fluid outlet. As such, advantageous use can be made of gravitational forces to dispense the fluid mixture. The dispensing unit is in particular advantageous for adding flavour or other additives to water. Preferably the supply fluid comprises water or filtered water. This can be tap water or bottled water or carbonated water. The fluid supply connection may comprise a solenoid valve, in particular a 3/2-way solenoid valve and/or a 3-1-way solenoid valve. It is possible that the speed of waterflow is internally measured within the dispensing unit. An additive supplied by the additive supply can comprise for example a flavour, a colourant, a vitamin additive or the like. The additive is preferably also a fluid, but an additive in other forms are also conceivable. If multiple additive supplies are applied it is possible to combine multiple additives into one fluid mixture. The additive supply may be connected to a pump and additive reservoir for supplying the additive to the additive supply. The type of pump used for displacing the additive is preferably a gear type pump. However, a bi-directional pump in general may also be used, as well as a peristaltic pump. In case a gear type pump is used, it is preferred that the gears thereof are about 5 mm diameter, in particular 4 mm diameter. This size turned out to provide for a consistent flow rate, regardless of the phase. Typically each additive supply is positioned above or upstream of the fluid outlet. The additive supply can be positioned in proximity to, preferably not in, the mixing chamber. The additive supply can be positioned on top of the mixing chamber, or for example outside alongside the mixing chamber. In case multiple additive supplies are present in the dispensing unit, they are preferably arranged in pairs or at a circumference around the mixing chamber at an equal mutual distance from one another. In a preferred embodiment the additive supply is arranged downstream and/or below of the fluid supply connection.

The dispensing unit according to the present invention also comprises a disinfection element, for disinfecting at least a portion of the mixing chamber and/or the at least one additive supply, in particular at least during use. Preferably the dispensing unit comprises at least three additive supplies, more preferably at least six, preferably arranged around the central axis, more preferably equally spaced around the central axis. If six additive supplies are provided, it is conceivable and preferably that said six additive supplies are arranged in three pairs of two additive supplies. It is preferred if the additive supply or supplies comprises at least a directional component oriented towards a central axis of the dispensing unit. However, it is particularly preferred if the additive supply or supplies are oriented towards the mixing body if applied, in particular towards the mixing surface of the mixing body. To this end, it is preferrable if the additive supply or supplies are substantially orthogonal with respect to the mixing surface. Due to users touching parts of the dispensing unit, it is possible that bacteria get into the dispensing unit. Additive, and more in particular flavour additives are more prone to bacterial growth. Flavour additives often comprise sugar, which may increase the bacterial spreading speed. A disinfection element can prevent growth of the bacteria and can also kill and/or inactivate them. This improves the hygiene of the dispensing unit according to the present invention. Preferably the disinfection element is positioned outside of the mixing chamber. An example thereof is that the disinfection element is positioned (centrally) above the mixing chamber. It is preferred that the disinfection element is oriented towards the mixing chamber. It is possible that the disinfection element also disinfects, a portion of, the supply fluid, for example water or filtered water, the additive and/or fluid mixture during use. The disinfection element may be configured to disinfect at least portion of the additive supply, in particular the tip of the additive supply, where the additive leaves the additive supply. If applied, disinfecting the additive supply prevents bacterial back growth or bacterial bloom into the additive supply. Bacterial bloom is a typical problem occurring in dispensing systems with open type of additive supplies. Open type of additive supplies should be understood as additive supplies with an opening for providing of additive, which remains opened in a state in which no additive is supplied. For example a pipe of a certain length with an inner channel. In such type of additive supplies it may occur that droplets remain on the outlet of the supply. This may cause a source for bacterial infection and possibly bacterial bloom. The disinfection element prevents bacterial growth within the dispensing unit, thereby improving its hygiene. It is conceivable that the at least one additive supply closes when no additive is supplied. Hence, preventing, or reducing an opening for bacterial growth. Preferably, the additive supply closes impermeably. Yet, it is also conceivable that the additive is, at least slightly, retracted after use, preventing droplets from forming on the tip of the additive supply. The additive supply may inject and/or insert the additive into the mixing chamber. Besides droplets and dripping, the open channel type of additive supply in general is more exposed to air. Increasing the contaminable surface, as well as risks of drying & solidification of the additive such as flavour (and risks of blockage). Besides this, having unpredictable droplets may result in undesired mixing with the supply fluid, making supply fluid without additive difficult, which is an often occurring problem in the field. Finally, droplets falling from an open channel are often backed by air inside the channel. This will affect the reliability of the mixing ratio for the next mixed fluid with that additive.

The dispensing unit may allow a user to select the desired type of supply fluid (bottled water, tap water, cooled water, carbonated water, filtered water or a combination thereof) and optionally one or more additives. The dispensing unit may also comprise a control unit for controlling the amount of additive and amount of supply fluid to get the desired mixing ratio in the fluid mixture. It is possible that the fluid mixture comprises the supply fluid only and that no additive is chosen by the user. This is in particular achieved by means of the additive supply according to the present invention. Since the supply fluid is physically separate of the additive supply it is possible to dispense a supply fluid without additive. In particular, the supply fluid provided into the mixing chamber does not contact the additive supply. Therefore, when no additive is opted for, the supply fluid does not unintentionally rinse of residuals on the additive supply. Moreover, the specific type of additive supply further contributes to this aspect. It is imaginable that the fluid distribution element is configured to direct the supply fluid into the mixing chambers whilst not directing the supply fluid towards the additive supply.

The dispensing unit may further comprise at least one filter unit for filtering the fluid, in particular wherein the at least one filter unit is positioned downstream or upstream of the fluid supply connection.

When the dispensing unit is in use, the disinfection element may typically be activated or not activated. The term in use should at least be understood as a supply fluid flowing through the dispensing unit. Also in use may be understood as a sensor, such as a PIR sensor (if applied), has detected presence of a potential user. The term in use does not necessarily require an additive to be injected by the additive supply, since a user may also opt to dispense carbonated water, filtered water, additionally, the temperature of the supply fluid may be chosen. The choices are preferably continuous, meaning that a user may opt for a desired amount of carbonation, or any temperature within a predetermined range, such as between 0 degrees and 100 degrees. It is preferred that the disinfection element is active during, and in particular also just before the use of the dispensing unit. The disinfecting unit is in particular activated when no user is detected in proximity of the dispensing unit. In particular, the disinfection element is activated on an interval basis, when no presence is detected. Having the disinfection element, in particular a UV(C) module active when a user is present may result in scenarios where the user is (directly) exposed to the UV(C) light. Direct exposure with a UV(C) module may not necessarily result in permanent harm, but is still safer to avoid, therefore the PIR sensor, if applied, is used as a condition for activation if the disinfection element. Moreover, it has turned out the UV(C) module has better disinfecting properties to the (dry) surfaces rather than the liquids or wet surfaces.

According to a preferred embodiment, the dispensing unit comprises at least one flush outlet, in particular a cleaning flush outlet. The flush outlet is in particular used in case a cleaning module is inserted, or a cleaning chamber is provided. The supply fluid connection may, such via the cleaning module, be connected to the flush outlet. Hence, allowing supply fluid to internally rinse the additive supplies, in particular duckbills, and leave the dispensing unit from the flush outlet. It is conceivable that a valve is arranged for switching between a first state, wherein the supply connection is fluidly connected to the mixing chamber and/or cleaning chamber, and a state wherein the flush outlet is connected to the mixing chamber and/or cleaning chamber. Said valve may further allow for switching between different types of supply fluid to be supplied to the mixing chamber. Hence the dispensing unit may comprise a plurality of fluid supply connections, wherein a valve is configured for fluidly connecting one fluid supply connection to the mixing chamber.

In a preferred embodiment of the present invention, the at least one disinfection element comprises at least one UV light source, in particular a UV-C light source with a wavelength situated between 250 nm and 280 nm, in particular between 260 nm and 275 nm. This wavelength has good disinfection properties for disinfection of fluids such as a type of (filtered) water, and also for disinfection of surfaces. UV light as a disinfection element is in particular advantageous for easy disinfection since it can reach all parts that need or desire disinfection more conveniently compared to mechanical cleaning. Preferably the dispensing unit comprises a single disinfection element comprising a UV light source, such as, but not limited to a UV-C light source. The light source, if applied, is preferably positioned such that the light is emitted from the source in a downward direction. It is particularly preferred that the light source, or at least a light emitted by said light source, is arranged in line, preferably substantially coinciding, with the central axis of the dispensing unit. It has turned out that this particular position allows for a good disinfection of the interior volume of the dispensing unit using only a single UV light source. Preferably the mixing chamber is positioned downward from (below) the disinfection element, such that the light emitted is aimed towards the mixing chamber. Thus, allowing the mixing chamber to be disinfected. Alternatively, the disinfection element may be positioned next to, such as on the same height, the mixing chamber. In that case, the light source is positioned such that the light direction is directed towards the mixing chamber and hence the disinfection element arranged at an angle with respect to the central axis of the dispensing unit. The light source as disinfection element, if applied, is preferably positioned such that UV light is not emitted from the fluid outlet. This may e.g., be achieved by means of a stationary mixing body (if applied). Said mixing body may at least partially obstruct or block a light flow path of the emitted UV light. Preferably, at least a portion of the light emitted is directed towards at least one additive supply. Hence, allowing the disinfection element to also disinfect the additive supply. This is beneficial since additive supplies are prone to bacterial bloom.

If a light source as disinfection element is applied, it is preferred that the disinfection element comprises a light guiding element, wherein the light guiding element is configured for guiding at least a part of the light emitted by the UV light source towards mixing chamber and/or additive supply. This is advantageous for an effective disinfection, since the mixing chamber and/or additive supply are the places where most disinfection need arises. It is also conceivable that no light guiding element is provided, in which case the light source, such as the UV(C) light source is located at an ideal illumination location. Said idea illumination location for example being a location allowing a light emitted by said light source to directly be emitted into the mixing chamber and/or onto a mixing body, and/or onto the additive supply. Preferably, said ideal illumination location at least directly emitting light to at least one of aforementioned elements, and optionally on one or more elements in for example an indirect manner. Direct illumination would reduce the amount of elements required for a proper disinfection, contributing to a lower cost of the dispensing unit. Yet, also no light losses occur which may be occurring in the light guiding element and therefore a more efficient disinfecting may be established. It is conceivable that the light guiding element (and/or disinfection element), preferably at a side facing the mixing chamber comprises a cover plate. Said cover plate preventing liquid from entering the light guiding element. Said cover plate is preferably composed out of glass, in particular clear glass, such as to allow UV light to pass through, and reach the mixing chamber, but preventing liquid from entering the light guiding element. A particular glass that has proven to be suitable for the wavelengths of the used UV light is (JGS1) quartz glass.

Using a light guiding element has the advantage that the light from the disinfection element can be easily concentrated and/or guided. For example, the light may be guided towards a mixing body, if applied. The mixing body thereby being disinfected by the light and preferably also blocking the light with respect to the fluid outlet. It is also possible that the light is guided towards a reflective surface, thereby increasing the reach for disinfection. Reflective surfaces can be part of the mixing body and are preferably positioned such that light from the disinfection element is reflected towards for example the walls of the mixing chamber, and/or the additive supplies, and/or the mixing body itself. In particular the end tips of the additive supplies where the additive leaves the additive supply can be disinfected. This prevents bacterial back growth into the additive supply.

In a preferred embodiment, the dispensing unit according to the present invention the disinfection element is configured for simultaneously disinfecting at least a part of the mixing chamber and/or at least a part of the at least one additive supply, optionally of a supply fluid. It is also conceivable that other parts that are present within the mixing chamber are disinfected by the disinfection element simultaneously. This is in particular advantageous for a fast and in particular efficient disinfection, such that repetitive use of the dispensing unit can be performed easy without increased risk of bacterial bloom. In between use of the dispensing unit the disinfection element can be activated and by disinfecting several items simultaneously it is possible to quickly provide multiple users of a desired dispensed fluid mixture.

To prevent back growth of bacteria even further, it is advantageous that there is little or no drops of supply fluid and/or additive residing within the dispensing unit. Therefore, it is advantageous that at least one, preferably each, additive supply comprises at least one backflow-prevention element. Especially suitable for this purpose is a duckbill valve. Other examples that can be applied to prevent backflow are a check valve, an atmospheric vacuum breaker, a double check valve assembly, a dual check valve. The duckbill valve may for example be combined with a gear pump for transporting the additive. The gear pump easily allows for transporting additive in both directions (from and towards the mixing chamber). Hence, it may allow for opening and/or closing the duckbill valves. Said duckbill valve can be predictably closed for example using a bi-directional pump such as a gear pump or, peristaltic pump, and preferably in combination with gravity, preferably a height difference of at least 80 cm, providing the minimal negative pressure required to keep a duckbill closed once the inertia of the additive fluid is overcome be reverting the direction of the pump.

According to a preferred embodiment an air gap is provided in between the additive supply and the mixing chamber. In that case, in the dispensing unit according to the present invention, the at least one additive supply can be positioned at a distance from the at least one mixing chamber. This distance creates an air gap, wherein air is in between the additive supply and the mixing chamber. The additive supply may not be in direct contact with the supply fluid and/or fluid mixture. In this case not direct contact means, that during normal use in dispensing state the supply fluid does not get into touch with the additive supply. Furthermore, there can be no direct path from the fluid outlet towards the additive supply. It is therefore possible to have a continuous stream of additive supplied towards the mixing chamber, but after supplying the additive, there is an air gap in between the additive supply and the mixing chamber.

There may be a difference between operational states of the dispensing unit, in particular when an air gap is present during use of the dispensing unit. It is possible that during a rinsing or a cleaning state of the dispensing unit, the air gap is temporarily bridged to allow cleaning and/or rinsing liquid to reach the additive supply for cleaning or disinfecting purposes.

Within the mixing chamber of the dispensing unit according to the present invention an additive is mixed with a supply fluid into a fluid mixture. An example of a fluid mixture can be defined as a fluid stream with uniformly distributed additive. Where the term mixing is used, also diluting, blending may be understood. As long as the fluid mixture as a substantially uniform appearance, in terms of distribution of the additive within the supply fluid.

Mixing in the mixing chamber may be achieved by injecting or dropping an additive into the supply fluid in the mixing chamber. Due to the crossing of the flow of both fluids they will mix into a fluid mixture. According to an advantageous embodiment, the dispensing unit comprises a mixing body for mixing of the additive and supply fluid. The mixing body may receive both a portion of the supply fluid and optionally a portion of the additive for creating a fluid mixture. In a preferred embodiment, the dispensing unit comprises at least one mixing body, preferably a stationary mixing body. In this respect, stationary should be understood as the mixing body as such is not rotating in order to mix the additive and supply fluid. Preferably, said mixing body is at least partially accommodated in the at least one mixing chamber. Preferably, the at least one mixing body shares a central axis with the central axis of the dispensing unit. Preferably, at least a portion of the supply fluid is oriented towards the mixing body.

The mixing body may comprise at least one, preferably a plurality of, mixing surface. Said mixing surface preferably oriented such as to receive part of the supply fluid and at least one additive. The mixing surface may change the speed of the supply fluid and/or the at least one additive, in particular decreasing the speed of the supply fluid flow and/or additive flow. The mixing surface further can have the effect of changing the direction of the flow. Decreasing the speed of the additive flow into the mixing chamber turned out to be advantageous. It allows for using the dispensing unit for a broader range of dispensing flows. Mixing of the additive and supply fluid may be achieved by means of said mixing surface. To this end, an additive on said mixing surface may be rinsed by the supply fluid running over or aimed towards the mixing body.

For mixing it is advantageous if the additive supply can be positioned and/or oriented such that the additive hits a mixing surface of the mixing body, preferably in a straight line. Therefore, in an embodiment, at least one additive supply, preferably each additive supply, is oriented in a direction towards a mixing surface of the mixing body, for allowing supplied additive to be at least partially mixed with the supply fluid in proximity of and/or on the mixing surface. Preferably, the number of additive supplies is equal to or less than the amount of mixing surfaces. In that way each additive supply can be positioned opposite of a mixing surface. This may prevent additives from mutually mixing on a mixing surface, which may cause the fluid mixture to comprise traces of an unintentional additive. That is, residue of the previously chosen additive may reside on the mixing surface, and cause the next beverage to comprise traces of the previous additive. In case mixed flavours are used, it is also possible that multiple additive supplies are directed towards the same mixing surface. However, it is preferred that each additive is oriented towards a separate mixing surface. Also, this may reduce the local increases in flow speed when dispensing the fluid mixture, in particular when multiple additives are chosen by a user. These local non-symmetric increases may contribute to splashing or a deflection of the resulting mixed beam, which is highly undesirable for users. Said mixing surface greatly contributes to eliminate or at least reduce said splashing.

During use, preferably the supply of additive will stop sooner than the supply fluid. This may result in the supply fluid to wash off potential residual additives from the mixing body. In this way the supply fluid will flush or rinse or neutralise the mixing body for a subsequent fluid mixture to be made. Another advantageous aspect of a preferred embodiment of the dispensing unit according to the present invention is that the disinfection element can be directed towards at least one surface of the mixing body for disinfecting at least a part of the mixing body. For example, the disinfection element is positioned centrally above the mixing body. This way the disinfection element can disinfect at least a part of the mixing body. For example, if UV light is applied, in particular UVC, which disinfects all parts of the mixing body where the UV light can reach the mixing body, an easy disinfection may be realized. The mixing body comprises mixing surfaces according to a preferred embodiment. The mixing body is preferably centrally positioned within the mixing chamber, in particular sharing a central axis with the dispensing unit. If multiple mixing surfaces are applied at least two mixing surfaces may mutually form a (consecutive) circumferential surface. Preferably, at least two mixing surfaces mutually enclose an angle. It may be conceivable that the mixing body comprises a central body, with at least one, preferably multiple, protruding elements. Said protruding elements may be formed by laterally protruding elements. Said laterally protruding element may be a fin, wherein surfaces of said fins, in particular surfaces facing away from each other, may form a mixing surface. It is preferred that the central body comprises at least three laterally protruding elements, in particular fins. Radially positioned at an angle of 120 degrees with respect to each other. Hence, allowing to form at least six separate mixing surfaces.

The dispensing unit may further comprise at least one dispensing grid, arranged at least partially inside the mixing chamber, preferably in proximity of the dispensing opening. However, it may be imaginable that the dispensing grid is arranged outside the mixing chamber. The dispensing grid may improve the stream of dispensed fluid, hence the mixed fluid. It has been found that the grid especially improves the dispense of liquid at a flow rate between about 0.5 to 2.4 L/min, especially 1.1 to 2.4 L/min. This enables a total dispense flow range of 0.5 to 3.5 L/min, preferably 1.1 to 3.5 L/min, more preferably 2.2 to 3.5 L/min, wherein the dispensing unit may allow for adequate dispensing over substantially the entire range. Preferably, and independent of whether a mixing body or a dispensing grid is provided, the fluid that is discharged via the discharge grid is a bonded fluid stream, in particular a bonded laminar fluid stream. Such a bonded fluid stream may be bonded over a distance of at least 5 cm, more preferably at least 15 cm, in particular at least 30 cm. Here, a bonded fluid stream may be understood as a straight stream, essentially without drop formation and without flooding of the mixing chamber. This allows to fill either a small glass, a carafe, bottles, or the like, independent of their height without drops or liquid landing besides the object to be filled. The dispensing grid may be provided in addition to, or as an alternative to the mixing body according to the present invention.

It is imaginable that the at least one dispensing grid comprises at least one annular ring and a plurality of spokes, said spokes connected to an inner surface of the mixing surface and the at least one annular ring. The spokes may be arranged at a downward angle, at least as seen from an internal surface of the mixing chamber towards the center of the dispensing grid. The angle of at least one, preferably all spokes is about 15 to 25 degrees. It is conceivable that at least one spoke comprises a first portion, extending from the interior surface of the mixing chamber towards the annular ring at a first angle, and a second portion extending from the annular ring towards the center of the dispensing grid at a second angle. The second angle may be bigger compared to the first angle. This may contribute to dispensing a solid stream of fluid. It is imaginable that the dispensing grid comprises a plurality of spokes, for example 3, 4, 5, 6, 7, 8, 9, or 10 spokes. Preferably the spokes are arranged at constant mutual angles. Preferably, the angle between two spokes is about 60 degrees.

The dispensing grid may comprise at least one core, wherein a side of the core facing away from the fluid supply connection is tapered, in particular cone-shaped. It is conceivable that the side of the core that is tapered or cone-shaped core has an angle of 25 degrees, in particular with respect to a horizontal plane. The tapering or cone-shaped part may reduce the amount of residue that remains on the dispensing grid. The tapered or cone-shaped portion may reduce the available surface tension such that less, and if at all smaller drops are formed. Where most dispensing grids are substantially planar, and have a substantially flat underside, this causes drips to form at random places. The tapered or cone-shaped portion guides residues of liquid towards the center of the dispensing grid. This causes the drips, if any drips at all, to migrate to the tip of the tapered or cone-shaped portion. It may also contribute to the instantaneous discharge of water droplets at the end of a dispensing session. This may prevent delayed drips, or random dripping of the nozzle, which is not desired. From a bacteriological point of view, but also from a (flavour)contamination point of view, it may be preferred that the amount of water retained within the nozzle is reduced. It is imaginable that at least a portion, preferably a central portion, of the dispensing grid is lower compared to the rest of the dispensing grid. At least lower when the dispensing unit is in use.

In order to provide for sufficient hygiene the at least one dispensing unit may be at least partially arranged in direct line of sight with the at least one disinfection element. Here, direct line of sight may be understood as the UV light emitted from the disinfection element to be able to directly illuminate the dispensing grid. Hence, it is conceivable that a transparent and/or translucent layer is situated between the disinfection element and the dispensing grid. The dispensing grid is provided such that the light emitted by the disinfection element travels, preferably exclusively, through light translucent medium(s). The dispensing grid may be arranged directly below, in the vertical direction, the disinfection element.

Preferably the mixing body may comprise a reflective surface, in particular the mixing surfaces are reflective surfaces, which reflect UV-light, if applied, in particular reflect UVC-light. The mixing body may further comprise a reflective surface for reflecting the UVC-light, if applied, towards other parts of the dispensing unit. Preferably said reflective surface is facing upwardly. This is advantageous for increasing the reach of the UV(C) light and thereby increasing the disinfection effect thereof. The mixing surfaces can be reflective. The reflective mixing surfaces may be oriented such that UV(C) light is reflected from the disinfection element towards the additive supply.

It is beneficial if there is close to no residual supply fluid or additive left on the mixing body, or on the mixing body surface. Therefore the mixing body may preferably comprise smooth surfaces. If smooth surfaces are applied, these can be easily cleaned by the supply fluid and it is less likely that residual fluids will remain.

The mixing body can have multiple shapes, for example but not limited to, a cross section which is substantially triangular, circular, oval, polygonal, starshaped, three-point-starshaped, tripod-shaped or rectangular. Preferably the mixing body is a stationary mixing body which is fixated within a central position within the mixing chamber. With central it is meant that with respect to the additive supplies the mixing body is placed in a central position within the mixing chamber. In a preferred embodiment, the stationary mixing body is substantially entirely (at least 90%) positioned downstream with respect to the additive supply.

It is beneficial if an element is present in the dispensing unit which ensures that a controlled stream of fluid is dispensed. An aerator can be used to create a controlled stream of fluid. However, aerators typically cannot handle UV(C) light well. So, if UV(C) light is applied as a disinfection element, aerators are preferably not used. Furthermore aerators typically only provide a stream downwards. If an aerator would be used in the dispensing unit, the sides of the mixing chamber would not be sufficiently flushed with supply fluid to take away any residuals.

In a preferred embodiment, the dispensing unit further comprises at least one fluid distribution element. Preferably, the at least one fluid distribution element comprises a plurality of fluid distribution channels, each channel for distributing a portion of the supply fluid into the mixing chamber. The fluid distribution element is preferably positioned in between the fluid supply connection and the mixing chamber. In a preferred embodiment, the light guiding element of the disinfection element, fluid distribution element, mixing chamber and the mixing body share the same central axis.

Preferably, a space between the at least one fluid supply connection and the fluid distribution element is as small as possible. This may ensure there is as little as possible supply fluid of the (possibly) wrong type present or retained in the dispensing from a previously dispensed liquid. The fluid retained in said space may between use be disinfected by periodic activation according to the present invention. That is, a 3/2 way solenoid valve (if applied) preloads the correct type as the user selects its preferred supply type. Hence, the amount of liquid residing in this space is as small as possible, and therefore less unfresh or wrong liquid resides which may be dispensed during a subsequent dispensing session. Due to less liquid residing after dispensing, it is less likely that the dispensing unit will have post-dispensing drips hanging from the outlet. The latter is a severe problem in particular when dispensing sparkling water, as the dissolvability of CO2 in water is dependent on pressure and low temperature. Both are no longer present after the valve, which will cause CO2 bubbles to form between said solenoid and the fluid distribution element, pushing out water and causing dripping. Due to the reduced space between the fluid supply connection and the fluid distribution element, the amount of drips after dispensing is significantly reduced. Yet, another function of the flush outlet may be to rinse out any residual liquid, to ensure the dispensed supply fluid is directly of the correct type, freshly filtered, and of the right temperature. The space is preferably at most 4.5 mL, preferably between 1.0 mL and 4.5 mL.

The distribution element may be provided with fluid distribution channels which distribute the supply fluid into the mixing chamber in a controlled way. The distribution element may be circular, preferably spanning over a passage in between the fluid supply connection to the mixing chamber. In addition, the fluid distribution element may comprise at least one, preferably a plurality of, fluid guiding fin(s), said guiding fin located upstream of the fluid distribution channels, for allowing to establish a substantially laminar fluid flow. A laminar fluid flow is advantageous for a smooth and controlled flow through the distribution element and for a controlled mixing in the mixing chamber. Moreover, it highly benefits the resulting angle of the fluid mixture beam and laminarity thereof, preventing splashing, breaking, or missing the user's fluid retaining element. A controlled flow increases the reliability of the by the user chosen mixing ratio of supply fluid additive. Yet it is also conceivable that the at least one fluid supply connection may be connected to the mixing chamber via a fluid supply channel. Said fluid supply channel may comprise one or more fluid guiding fins, for reducing the amount of turbulence in the supply fluid flow. In this particular case, the at least one fluid distribution element may define the end of the fluid supply channel.

Preferably the distribution element comprises multiple fluid distribution channels for distribution of the supply fluid, which fluid distribution channels, if applied, are at least partially inclined downwardly. In particular the distribution channels are at least partially defined by a straight channel, and/or straight through-hole in the fluid distribution element.

Preferably, the fluid distribution element may comprise at least one inwardly oriented fluid distribution channel, wherein each inwardly oriented fluid distribution channel is inclined inwardly with respect to the central axis of the distribution element and/or central axis of the dispensing unit. The inwardly inclined fluid distribution channels, if applied, are preferably directed towards a central part of the mixing chamber. In particular, the inwardly inclined fluid distribution channels may be directed towards the (stationary) mixing body, for example towards a particular mixing surface of said mixing body. The inwardly inclined fluid distribution channel may be inwardly oriented under an angle in the range of substantially 20-30 degrees, preferably 25 degrees with respect to a vertical axis. Said vertical axis being the central axis preferably. The inwardly inclined fluid distribution channels are preferably divided such that they correspond to the mixing body applied in the embodiment. For example, the inwardly inclined fluid distribution channels may be divided equally around the circumference of the distribution element. It is also conceivable that the inwardly inclined fluid distribution channels are aimed towards a particular mixing surface of the mixing body. This may allow for an enhanced mixing, and preventing residual additive to remain.

It is further preferred that the fluid distribution element comprises at least one outwardly oriented fluid distribution channel. Each outwardly oriented fluid distribution channel is preferably inclined outwardly with respect to the central axis of the distribution element or dispensing unit. This inclination can be under an angle in the range of substantially 20-30 degrees with respect to the vertical axis, in particular substantially 25 degrees. Said vertical axis being the central axis preferably. The outwardly inclined fluid distribution channels, or through holes, contribute to a controlled stream of supply fluid downstream of the distribution element. The outwardly inclined fluid distribution channels can be directed towards the outer walls of the mixing chamber. Preferably, the outwardly inclined fluid distribution channels may be divided equally with respect to the circumference of the distribution element. This is advantageous for obtaining a uniform flow or stream of fluid mixture towards the bottom opening of the mixture chamber and consequently directly or indirectly towards the fluid outlet.

In a preferred embodiment, the centerlines of at least two of the plurality of fluid distribution channels mutually enclose an angle. The distribution element may comprise both inwardly and outwardly inclined distribution channels, or through holes. In a preferred embodiment about ⅓ of the fluid distribution channels are inclined inwardly and about ⅔ of the fluid distribution channels are inclined outwardly. It is particularly preferred that the inwardly inclined distribution channels and outwardly inclined distribution channels are arranged alternately around the circumference of the fluid distribution element. Alternately may for example be, two outwardly, one inwardly, two outwardly, one inwardly etc. Preferably, the total number of fluid distribution channels accumulates to about 16, 18, 20, or 22. However, different amounts are also conceivable. The number of fluid distribution channels, and preferably their cross sectional area, are matched to an intended flow rate range. Besides this, fluid distribution channels that are too small have a risk of (partial) blockage due to production tolerances or scale build-up, whilst fluid distribution channels that are too big might not provide enough surface tension to prevent post-dispense dripping of retained water. An ideal surface area of a fluid distribution area is about 0.025 cm2 , preferably between 0.01 cm2 and 0.035 cm2.

It is beneficial when at least one additive supply is inclined in a direction towards the mixing chamber. Preferably the inclination or angle is such that a centerline of the additive supply mutually encloses an angle with a central axis of the mixing chamber. The centerline of the additive supply, for example a duckbill valve, is preferably positioned under an angle with respect to the vertical axis (central axis of the mixing chamber) in the range of substantially 35-55 degrees, more preferably 38-52 degrees, even more preferably 39-51 degrees. The flow of additive may have at least one directional component in the direction of the centerline of the additive supply. The flow of additive supplied by the additive supply will thus hit the mixing body in the mixing chamber, if applied, under an angle. If a Duckbill valve is applied, the horizontal deviation, may be in the range of −5 to 5 degrees, preferably −3 to 3 degrees.

Preferably, the dispensing unit according to the present invention comprises at least one sensor, in particular a passive infrared sensor (PIR sensor), wherein said dispensing unit is configured for activating at least part of the dispensing unit upon detecting presence of a user (or a fluid retaining element) by the at least one sensor, in particular for activating at least one disinfection element when no presence is detected. The latter since it is a goal not to disinfect the fluids, but the dispensing unit, e.g., structural components such as the mixing body, itself. Preferably, the disinfection element is periodically activated, in particular when it is known that it is safe to do so. Periodically may for example be 10 to 30 seconds every 30 to 60 minutes. This would prevent bacterial growth on surfaces and retained fluids. The nozzle is designed to allow proper drying of all surfaces by exposure to air, further preventing the risk of bacterial bloom. Based on detecting a presence, the dispensing unit preferably activates an indicator for indicating an operational status, and/or internally flushing water, for example to precool its tubing, and/or stopping UV(C) disinfection if interference by a user is possible. Other types of sensor that can be used are sensors that can be used to detect presence of a person or motion of a person. The advantage of activating the disinfection element no user is present is that the disinfection element can disinfect the dispensing unit whilst preventing the user may come into direct contact with the UV(C) light. The user therefore does not have to wait unnecessary for disinfection prior to mixing and dispensing. Said sensor may further activate a presence indicator, for example a coloured (LED) light, which indicates that presence was detected and that the dispensing unit is ready for use. It is conceivable that said (LED) light source may indicate various stages of the dispensing unit, such as a dispensing state, wherein fluid mixture is dispensed, a cleaning state, wherein an interior is being cleaned, or the like. Preferably, said stages are indicated by means of differently coloured lights. Preferably, the sensor is configured for detecting approximately 50 cm in front of the dispensing unit, as it is then able to detect a person's feet, or higher parts of their body if they are closer, or a fluid retaining element.

The speeds of flow of supply fluid and additives can influence whether the fluid mixture can have a uniform distribution of additive in the fluid mixture. Tests have shown that it is beneficial when the speed of flow of the supply fluid is controlled in a certain range.

Preferably the supply fluid connection is configured for providing a flow of supply fluid, wherein the speed of flow of supply fluid from the supply fluid connection is in the range of 1.5 L/min-4.0 L/min, preferably 2.0 L/min-3.0 L/min.

Typically there is less additive than supply fluid in the fluid mixture. The speed of flow of additive is preferably less or equal than the speed of flow of the supply fluid. The additive supply can be configured for providing a flow of additive, wherein the speed of flow of additive from the additive supply is in the range of 80-350 mL/min, preferably in the range of 87-330 mL/min.

The mixing chamber may be part of a mixing module. The mixing module is preferably removable. The removable mixing module, if applied, is removable or insertable into a part (of the housing) of the dispensing unit. This may e.g., be realized by co-acting coupling elements, such as a screw or click connection. Said mixing module may comprise an outer threaded part, which may co-act with an internal part of the dispensing unit, in particular a housing of the dispensing unit. Preferably, said co-acting coupling elements defining an end position of the mixing body. In particular wherein at this end position, at least one, preferably all, mixing surface are opposing an additive supply. This has the advantage that the mixing module can be easily replaced in case of end of lifespan, malfunctioning, or the like. It also has the advantage that by taking the mixing module out of the dispensing unit, it is easier to reach other components of the dispensing unit for maintenance and allows separate cleaning of the mixing module. Alternatively, the mixing chamber can form an integral part of the dispensing unit. The mixing chamber can optionally have the same central axis as the central axis of the dispensing unit itself.

Said removable mixing module preferably comprises a stationary mixing body, which mixing body may be integrally formed within the mixing chamber. However, it is also imaginable that the mixing body is a replaceable mixing body, which is mounted in the mixing chamber. Typically, the mixing module comprises a funnel shaped mixing chamber wherein the top of the funnel forms a wide opening. Said wide opening may comprise a distribution element or be in close proximity to a distribution element as described above. The lower part of the funnel comprises an opening for dispensing of the fluid mixture formed within mixing chamber towards the fluid outlet. Preferably the fluid outlet is positioned in a straight line below the lower funnel opening.

An advantage of the dispensing unit according to the present invention is that due to the disinfection element it requires low cleaning maintenance. However, sometimes additional cleaning of parts of the dispensing unit may be desired. To this end, it is possible that the dispensing unit according to the present invention comprises at least one cleaning module. Said cleaning module may define a cleaning chamber for cleaning of internal parts of the dispensing unit. Preferably, said cleaning module, in particular the cleaning chamber internally directs the supply fluid towards the additive supply, in particular an additive supply outlet, and/or disinfection element and/or fluid distribution element, if applied. In this embodiment, the supply fluid may be a cleaning fluid. Preferably which cleaning module is inserted into or insertable into the dispensing unit. If applied, said inserted cleaning module may be configured to define a cleaning chamber for cleaning the additive supply and/or a part of the disinfection element and/or distribution element, if applied. The cleaning chamber may at least partly be defined by the walls of the mixing chamber. Preferably the cleaning module and mixing module are interchangeable. If the mixing module and cleaning module are interchangeable, the mixing chamber may function as a cleaning chamber. The cleaning module may be removed and/or inserted via co-acting coupling elements configured for coupling the cleaning module and the dispensing unit. Preferably, the same co-acting coupling elements are used for removing and/or inserting a mixing module. As such, a modular assembly may be provided, wherein one may easily interchange the mixing module and cleaning module. The dispensing unit according to the invention comprising the (removable) cleaning module does not necessarily require the presence of the mixing chamber according to the invention. Preferably, the cleaning module, or mixing chamber, is situated at least partially above the location of the mixing chamber (if present). The cleaning chamber should be positioned such that the supply fluid is directed towards the elements to be cleaned.

The supply fluid may be used as a cleaning fluid. In particular water as supply fluid is a suitable cleaning fluid. In this case, the, preferably removable and/or insertable, cleaning module may comprise at least one curved surface for redirecting the supply fluid from the supply fluid connection towards elements and/or parts to be cleaned, such as the additive supply and/or disinfection element when inserted. In a preferred embodiment, the cleaning module comprises a double curved surface, for example a circular surface with a bowlshaped curved recess in the middle and a ringshaped curved recess around said bowlshaped surface. In this embodiment each curve (bowlshaped curved recess and ringshaped curved recess) redirects the fluid flow in several directions, at least towards a component to be cleaned. Preferably, at least one curved surface is configured to receive supply fluid from inwardly inclined fluid distribution channels, and at least one curved surface is configured to receive fluid from the outwardly inclined fluid distribution channels, if applied. As such, both curved surfaces may direct a portion of the supply fluid in a particular direction.

The cleaning module can be positioned such into the dispensing unit that the cleaning module partially blocks the fluid outlet. This has the advantage that it is prevented that the cleaning fluid (optionally the supply fluid as cleaning fluid) runs straight down towards the fluid outlet, but the fluid outlet remains functioning as the outlet for the cleaning fluid used.

In a preferred embodiment, the dispensing unit according to the present invention comprises at least one control unit. Said control unit can be configured for controlling a predetermined mixing ratio of the amount of supply fluid and the amount of at least one additive. For controlling the mixing ratio a flow rate sensor can be used in combination with the control unit. The control unit may use the flow rate sensor in determining when to stop the flow of additive and/or stop the flow of supply fluid to the mixing chamber. Preferably controlling the predetermined mixing ratio of the fluid mixture is based on sensor data, in particular flow rate data of the flow rate of the supply fluid and/or data of the flow rate of the additive obtained from a flow rate sensor. However, more preferably, a combination of a rotary encoder on the gear pumps to determine the rpm and calibration data on the fluid displacement per rotation is used to determine the flow rate.

The user of the dispensing unit may select a predefined mixing ratio and flavour. The flavour is chosen and configured out of at least one additive. A combination of more than one additive is possible, according to the desire of the user. It is also possible to select no flavour and only obtain supply fluid from the dispensing unit. In addition, the strength of the chosen flavour can be determined by the user. The selection made by the user is used by the control unit to control the supply of fluid supply and supply of additive to obtain the selected mixing ratio.

In a preferred embodiment, the control unit is configured to stop the supply of additive prior to stopping the supply of fluid supply, wherein the predetermined mixing ratio is maintained.

The control unit may be configured to delay the start of the additive supply with respect to the start of the flow of supply fluid. The delay of providing additive supply to the mixing chamber may be in the range of 100-200 ms, preferably substantially 150 ms.

Furthermore, the control unit may be configured to stop the supply of additive earlier than the supply of supply fluid. In particular it is advantageous to supply the supply fluid longer to flush away any residual additives within the mixing chamber. Preferably the supply of supply fluid continues for a short time, finding a balance between a controlled mixing ratio and flushing of the mixing chamber. The supply of supply fluid may continue for a time in the range of 200-300 ms, in particular 250 ms. It is conceivable that, in the case a dispensing grid is arranged, the control unit is configured to allow for a longer flush, for example in the range of 200-600ms. This may compensate for an increased water retention of a dispensing grid.

To maintain the predetermined mixing ratio and use a longer time for supplying the supply fluid than the additive, it is conceivable that the additive is supplied with a higher speed of flow rate in the beginning of the supply than at the end. In other words, the speeds of the flow rate of the supply of additive decreases over time. In order to allow the supply fluid to rinse the mixing chamber, it is preferred that the supply of additive is ended before the supply of supply fluid. Hence, allowing to rinse the mixing chamber with said supply fluid. In order to maintain the desired mixing ratio of the fluid mixture, the flow of additive supply is compensated for stopping in advance, for example by an initial higher flow.

The invention further relates to a beverage dispensing system comprising a dispensing unit according to the present invention. The beverage system according to the present invention has the same advantages as the dispensing unit as described above. The invention further relates to a method for dispensing flavoured water, comprising a dispensing unit, in particular a dispensing unit according to any of the claims 1-2, said dispensing unit comprising a mixing body with at least one mixing surface, comprising the steps of:
a) providing at least one water stream at least partially onto a mixing surface;
b) providing at least one additive by an additive supply, preferably after a delay with respect to step a), wherein each additive supply is positioned opposite of a mixing surface and the additive is provided onto the opposite mixing surface;
c) decreasing the speed of a part of the water stream of step a) and/or the additive supply of step b),

wherein step b) is ended prior to ending step a), rinsing additive residue on the mixing body.

Optionally, the step c) of the method according to the present invention is achieved by letting the part of the water stream and/or additive supply hit a mixing surface of the mixing body, at least a different part of a mixing surface, in particular wherein each additive stream is aimed at a different surface of the mixing body.

Using the method a mixing ratio can be guaranteed. For controlling the mixing ratio it is beneficial when the speed of flow of the stream in step a) is measured and/or when the speed of flow of providing the additive during step b) is controlled.

The same advantages as elaborated above with respect to the dispensing unit apply with respect to the dispensing system and the method according to the invention. The advantages are herewith incorporated with respect to the beverage dispensing system and method.

The invention further relates to a combination, or kit of parts, of a beverage dispensing system provided with a mixing module and a cleaning module.

The present invention will hereinafter be further elucidated based on the following non-limitative figures, wherein:

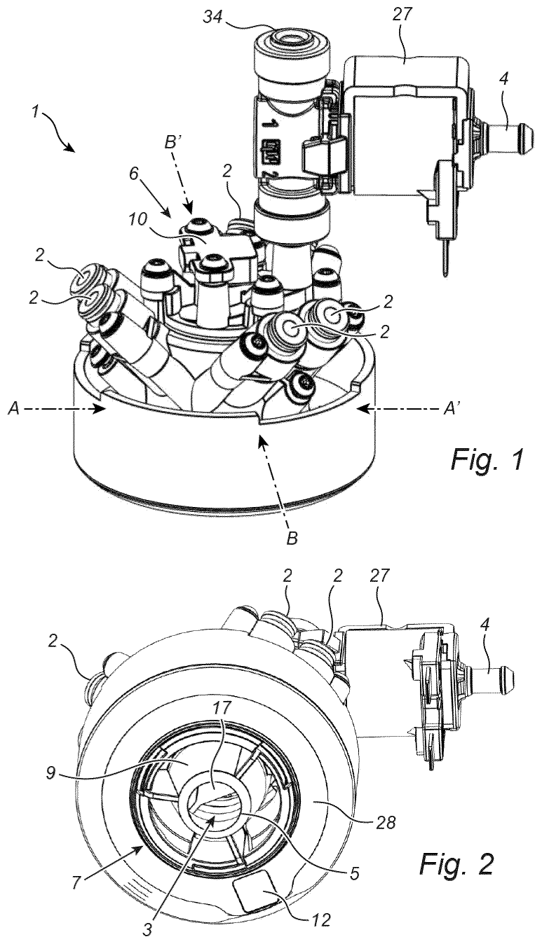

FIG. 1 shows a perspective view according to a first non-limitative embodiment of the dispensing unit;

FIG. 2 shows a second perspective view as seen from the bottom side of the dispensing unit as shown in FIG. 1;

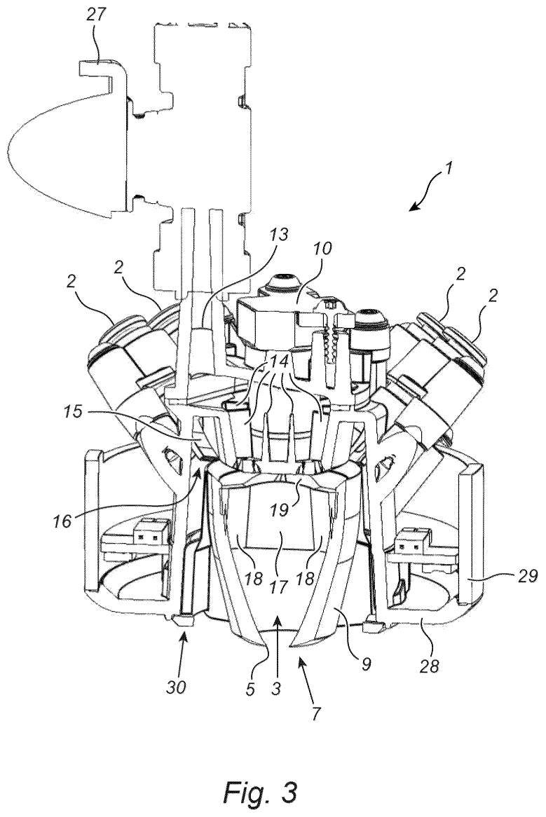

FIG. 3 shows a cross section in a vertical plane of the dispensing unit of FIG. 1 along line A-A′ as shown in FIG. 1;

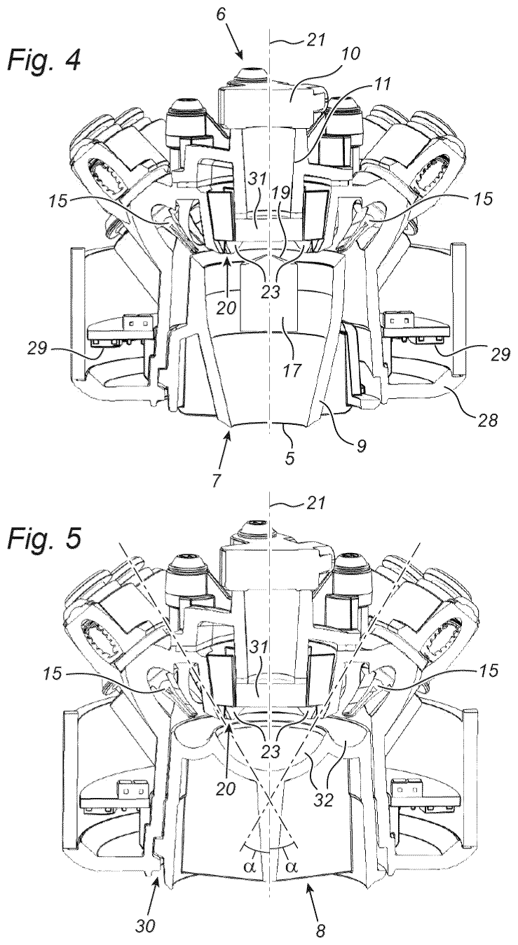

FIG. 4 shows a second cross section in a vertical plane of the dispensing unit of FIG. 1 along line B-B′ shown in FIG. 1;

FIG. 5 shows a cross section of a dispensing unit according to a different preferred embodiment of the invention;

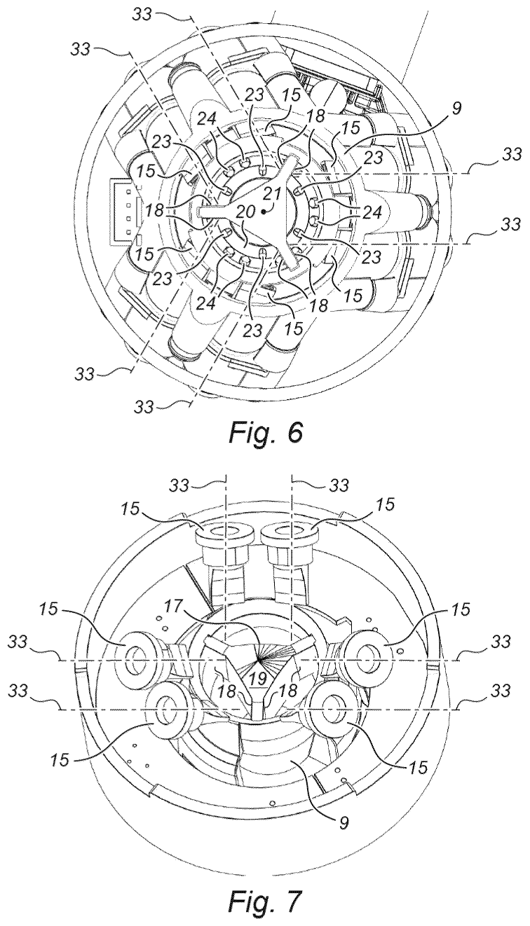

FIG. 6 shows a cross section in a horizontal plane of the dispensing unit, seen from a bottom side of the dispensing unit;

FIG. 7 shows part of the dispensing unit, seen from an upper side of the dispensing unit;

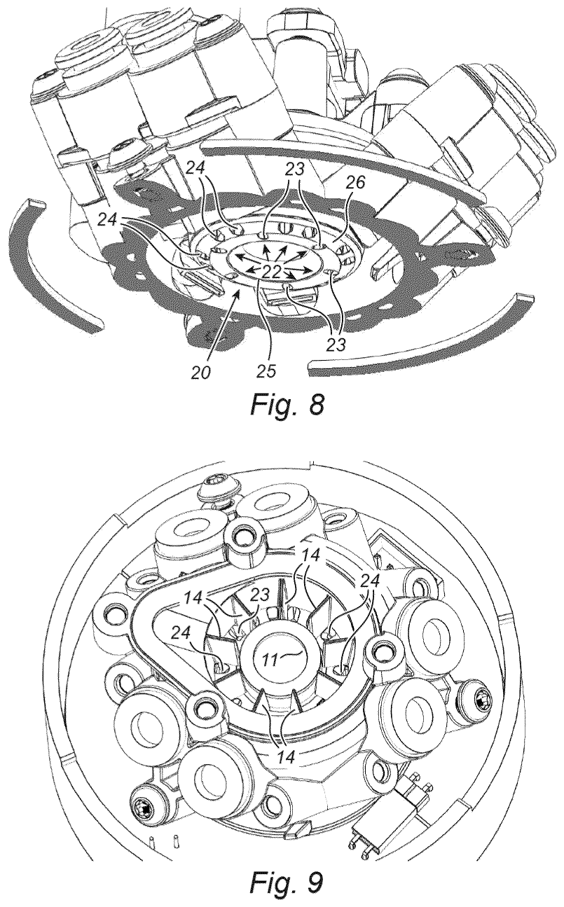

FIG. 8 shows a detailed view of a part of the dispensing unit as seen from an lower side according to the invention;

FIG. 9 shows a perspective top view of a portion of the dispensing unit;

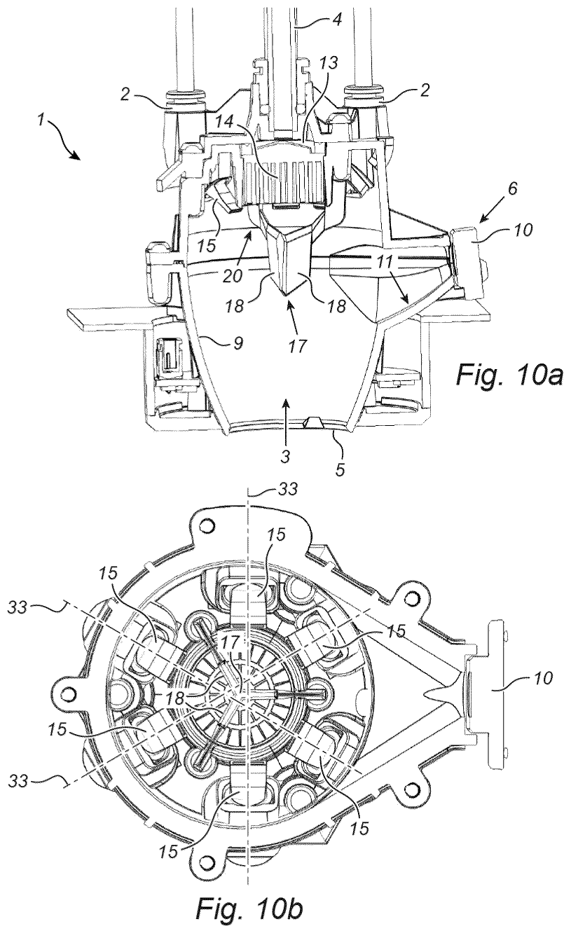

FIGS. 10a and 10b respectively show a cross section of a dispensing unit according to a different embodiment in a vertical and horizontal plane and;

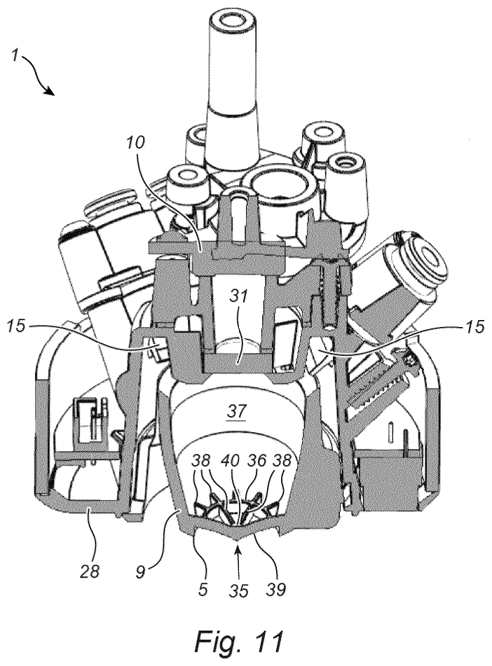

FIG. 11 shows a cross section in a vertical plane of a different embodiment of the dispensing unit.

FIG. 1 shows a first non-limitative embodiment of a dispensing unit 1 according to the present invention. The figure shows a perspective view of an upper side of the dispensing unit 1 according to the invention. Said dispensing unit 1 may be used for mixing an additive into a supply fluid. According to a particular preferred embodiment, the supply fluid is water, which may optionally be filtered and/or sparkling. The additive is preferably a flavour, such as strawberry, blueberry, chocolate, mint, or the like. It could be a liquid additive, however solid additives are also conceivable. In the latter case it is preferred that the solid additive is soluble, in particular water soluble. The dispensing unit 1 as shown in the figure comprises a supply fluid connection 34. Said supply fluid connection 34 may be connected to a supply fluid hose or cable or tube. In particular a water supply tube, such that water may be supplied towards the dispensing unit 1. In the latter case, water is the supply fluid. The flow of supply fluid may be controlled by means of a solenoid valve 27. As such an easy control of the supply fluid is established. The supply fluid is transported towards a mixing chamber 3 (FIG. 2). The solenoid valve is in particular a 3/2 way valve. In said mixing chamber 3 at least one additive may be supplied towards, and mixed with, the supply fluid to form a fluid mixture. In the non-limitative example of water as a supply fluid and strawberry-flavoured liquid as an additive, the fluid mixture may be conceived as a strawberry flavoured water. However, other examples are by no means excluded. The fluid supply connection 34 is situated above the mixing chamber 3. This allows the use of gravity and/or the supply fluid pressure for transporting of the supply fluid through the dispensing unit 1. At least one additive supply 2 is arranged on the dispensing unit 1 for introducing the at least one additive into the mixing chamber 3. The additive may be introduced directly or indirectly into the supply fluid, as will be discussed in more detail in the following figures. In this particular embodiment, three pairs of two additive supplies 2 are arranged on the dispensing unit 1. However, it will be apparent to the skilled person that alternatives thereto are also conceivable. In order to prevent and/or at least reduce a bacterial bloom in the dispensing unit 1 at least one disinfection element 6 is provided. Said disinfection element 6 in this example is formed by a UV module 10 for emitting a UV light, in particular a UVC light. The disinfection element 6 allows for disinfecting parts, in particular interior parts, of the mixing chamber 3 and the additive supply 2. Moreover, the dispensing unit 1 comprises preferably at least one flush outlet 4, in particular a cleaning flush outlet 4. The outlet is in particular used in case a cleaning module 8 is inserted, or a cleaning chamber is provided. The fluid supply 34 is, via the cleaning module, connected to the flush outlet 4. Hence, allowing cleaning fluid to internally rinse the additive supplies 2, in particular the duckbills 15, and leave the dispensing unit from the flush outlet 4. It is conceivable that the valve 27 is arranged for switching between a first state, wherein the supply connection 34 is fluidly connected to the mixing chamber 3 and/or cleaning chamber, and a state wherein the flush outlet 4 is connected to the mixing chamber 3 and/or cleaning chamber. Preferably, a space between the solenoid valve and the fluid distribution element is as small as possible. There is as little supply fluid of the wrong type present as possible, as the 3/2 way solenoid preloads the correct type as the user selects its preferred supply fluid type. Hence, the amount of liquid residing in this space is as small as possible, and therefore less unfresh liquid resides which may be dispensed during a subsequent dispensing session. Due to less liquid residing after dispensing, it is less likely that the dispensing unit will have post-dispensing drips hanging from the outlet. The latter is a severe problem in particular when dispensing sparkling water, as the dissolvability of CO2 in water is dependent on pressure and low temperature. Both are no longer present after the solenoids, which will cause CO2 bubbles to form between said solenoid 27 and the fluid distribution element 20, pushing out water and causing dripping. Due to the reduced space between the solenoid 27 and the fluid distribution element 20 the amount of drips after dispensing is significantly reduced. Yet, another function of the flush outlet 4 may be to rinse out any residual liquid, to ensure the dispensed supply fluid is directly of the correct type, freshly filtered, and of the right temperature.

FIG. 2 shows the same dispensing unit 1 as shown in FIG. 1, but showing a bottom side in a perspective view thereof. The bottom side of the dispensing unit 1 shows a part of the mixing chamber 3, wherein a stationary mixing body 17 is arranged. The bottom side of the device further comprises at least one fluid outlet 5. Said fluid outlet 5 allows to dispense the fluid mixture into a fluid retaining element, such as a holder. The fluid outlet 5 may at least partially be defined by an outer wall 9 of the mixing chamber 3. Said outer wall 9 may for example form a funnel to direct the fluid mixture into a solid stream. A user using this dispensing unit 1 may hold a glass, cup, bucket, bottle, or other fluid retaining element, below the fluid outlet 5 to fill his or her fluid retainer. In order to increase the ease of use of the dispensing unit 1, a sensor 12 is provided on the device, which in this particular embodiment is formed by a passive infrared (PIR) sensor 12. This sensor 12 allows for detecting presence of a possible user of the dispensing unit 1. As such, portions of the dispensing unit 1 may be activated. In particular it may be beneficial to activate e.g., the disinfecting unit 6. The disinfecting unit 6 is activated when no user is detected in proximity of the dispensing unit 1. This, as it ensures there is no risk of the user removing the being able to look directly inside the dispensing unit 1, into the UV-C module 6. However, it is also conceivable the disinfecting unit 6 is activated upon detecting presence of a possible user. In either situation the additive supply 2 and mixing chamber 3 are disinfected prior to dispensing a fluid mixture. Moreover, the sensor 12 may, directly or indirectly, activate a presence indicator. Said presence indicator may at least partially be formed by at least one LED. Preferably, wherein said LED is accommodated inside the dispensing unit 1, and emits a light through a (light) translucent material 28 on the bottom of the dispensing unit. This allows a potential user to visually recognize the dispensing unit 1 is active. According to this particular embodiment, the mixing chamber 3 is part of a removable mixing module 7. Said mixing module 7 may be connected in and/or removed from the bottom side of the dispensing unit 1. This may e.g., be realized via co-acting coupling elements, such as a screw or click connection. Said mixing module 7 may comprise an outer threaded part, which may co-act with an internal threaded part of the dispensing unit 1, in particular a housing of the dispensing unit 1.

FIG. 3 shows a cross section in a vertical plane of the dispensing unit of FIG. 1 along line A-A′ as shown in FIG. 1. The cross section allows to elucidate the interior of the dispensing unit 1 in more detail. The figure shows a supply fluid inlet channel 13. Said supply fluid inlet channel 13 guides the supply fluid towards the mixing chamber 3. Prior to entering the mixing chamber 3, the supply fluid flows through the supply fluid inlet channel 13, which comprises a section to form a more laminar flow. Said supply fluid channel 13 may to this end comprise at least one supply fluid guiding fin 14. Said fluid guiding fin is preferably oriented in a vertical direction, to form a vertical substantially laminar flow of supply fluid. A more laminar flow may yield a more predictable dispense from the dispensing unit 1. Alternatively or in addition, it is also possible that the fluid distribution element 20 comprises a section to form a more laminar flow. Said distribution element 20 may to this end comprise multiple guiding fins 14. The guiding fins 14 are positioned on the distribution element on the side facing away from the mixing chamber. Said fluid guiding fin is preferably oriented in a vertical direction, to form a vertical substantially laminar flow of supply fluid. A more laminar flow may yield a more predictable dispense from the dispensing unit 1. In particular a more predictable flow of supply fluid may be introduced in the mixing chamber 3. The mixing chamber 3 is shown to be at least partially defined and/or bounded by the outer wall 9. A stationary mixing body 17 is centrally located within the mixing chamber 3. Said stationary mixing body 17 comprises, according to this embodiment a plurality, in particular six, mixing surfaces 18. In the shown embodiment, each additive supply 2 is oriented towards a separate mixing surface 18. This prevents additives from being mixed, and as such may prevent additives to be mixed in case it is not desired. To this end, each additive supply 2 is preferably oriented towards a mixing surface 18. In particular a backflow prevention element 15 is oriented towards said mixing surface 18. The backflow prevention element 15 is in this embodiment formed by a duckbill valve 15. It has been found that the duckbill valve 15 significantly reduces the number of droplets residing on the additive supply. Said residual droplets form a great risk to potential bacterial blooming, which is undesired, and may in extreme cases contaminate an entire additive supply channel, situated upstream of the additive supply 2. Moreover, residual drops of additive may result in unintended mixing with supply fluid, resulting in the undesired addition of additive into the fluid mixture. The mixing surfaces 18 yet provide an additional functionality. The additive is injected towards said mixing surface 18, which reduces the speed of flow of the injected additive. By reducing the speed of flow of the injected additive, it was found that a wider range of fluid mixture outlet flows may be realized, whilst maintaining a properly mixed fluid mixture. Additionally, the mixing surface 18 improves the controlled distribution of the injected additive through the mixing chamber 3. Hence, it may reduce the amount of turbulence introduced into the fluid mixture. This greatly contributes to a more controlled flow of the fluid mixture. That is, the additive is injected onto the mixing surface 18, subsequently and/or simultaneously, the supply fluid flows over the stationary mixing body 17. The supply fluid flowing over the stationary mixing body 17 takes the additive that is injected towards the mixing surface 18 of the stationary mixing body 17 with it. Hence, the supply fluid rinses over the stationary mixing body 17 and simultaneously mixes with the additive on said mixing body 17. In an attempt to yet further reduce the amount of contamination of the dispensing unit 1 according to the invention, an air gap 16 is present between the additive supply, in particular the duckbill valve 15 and the mixing chamber 3. Hence, the duckbill valve 15 is positioned at a distance with respect to the mixing chamber 3. This allows for the duckbill valve 15 to be not in direct contact with the supply fluid and/or fluid mixture, in particular during use of the dispensing unit 1. On a bottom side of the dispensing unit 1, the co-acting coupling elements 30 establish a rigid connection between the removable mixing module 7 and the dispensing unit 1. Moreover, a bottom side of the dispensing unit 1 is preferably at least partially formed by a translucent material 28. Said translucent material 28 is understood to be translucent to a light emitted by an LED 29, which is situated above, and facing towards, the translucent material 28.

FIG. 4 shows a second cross section in a vertical plane of the dispensing unit of FIG. 1 along line B-B′ shown in FIG. 1. The figure indicates a central axis 21 of the dispensing unit. The disinfection element 6, which is formed by a UV module for emitting a UV(C) light, is centrally located in the dispensing unit 1. This allows for emitting a disinfecting light towards an internal part of the dispensing unit 1. In particular, this central arrangement of the disinfection element 6 allows for disinfecting the stationary mixing body 17 better. In particular since it allows to reach surfaces thereof which would in other arrangement not be disinfected. At least not conveniently by using a single disinfection element 6. Preferably, an upward facing surface 19 of the stationary mixing body 17 is at last partially reflective, or is configured for reflecting of light emitted by the UV module 10. As such, the upward facing surface 19 may redirect a portion of the UV light emitted by the UV module 10 towards at least one of the additive supplies 2, in particular the duckbill valves 15. In order to prevent the UV light from emanating from the fluid outlet 5, it is preferred that the stationary mixing body at least partially obstructs or blocks a light flow path. The light guiding element 11 may comprise an inward surface which is at least partially reflective, such that essentially all light emitted by the UV module 10 reaches the mixing chamber 3. However, in order to prevent fluids from contaminating the UV module 10 and/or the light guiding element 11, the light guiding element is covered by a cover plate 31. Said cover plate is preferably composed out of glass, in particular clear glass, such as to allow UV light to pass through, and reach the mixing chamber 3, but preventing liquid from entering the light guiding element 11. A particular glass that has proven to be suitable for the wavelengths of the used UV light is (JGS1) quartz glass.

FIG. 5 shows a cross section of a dispensing unit 1 according to a different preferred embodiment of the invention. According to this embodiment a cleaning module 8 is inserted into the dispensing unit 1. The cleaning module 8 may be removed and/or inserted via co-acting coupling elements 30 configured for coupling the cleaning module 8 and the dispensing unit 1. The cleaning module 8 may allow for internally cleaning the elements and/or components to be cleaned. In particular a cover plate 31 may be cleaned, as well as the duckbill valves 15. This may be realized via one or more curved surfaces 32. It is preferred that the cleaning module comprises two concentric curved surfaces 32, wherein each curved surface 32 is configured for redirecting a portion of the supply fluid towards a component to be cleaned. The supply fluid is used as cleaning fluid in this embodiment. According to this figure, the outer curved surface 32 is configured to redirect a portion of the supply fluid towards the duckbill valve 15. To this end, the dispensing unit 1 may comprise a fluid distribution element 20, which is configured for distributing the supply fluid in preferred directions. The fluid distribution element 20 may comprise fluid distribution channels 22, such as inwardly oriented fluid distribution channels 23. The inwardly oriented fluid distribution channels 23 shown in this figure direct a portion of the supply fluid towards the inner curved surface 32. However, when the mixing module is mounted (such as in FIG. 4) the inwardly oriented fluid distribution channels 23 direct the supply fluid towards the stationary mixing body 17, in particular allow a portion of the supply fluid to rinse and/or mix with the additive that is directed towards the mixing surface 18. Preferably, the inwardly oriented fluid distribution channel is at an angle α with respect to the central axis. A preferred range for the angle α about 20 degrees to 30 degrees.

FIG. 6 shows a cross section in a horizontal plane of the dispensing unit, seen from a bottom side of the dispensing unit. This figure shows the centerlines 33 of the additive supplies 15. According to this embodiment, each centerline 33 is directed to a particular mixing surface 18 of the stationary mixing body 17. The centerlines 33 of the additive supplies 15 preferably do not cross or intersect the central axis 21 of the fluid distribution element 20. This would otherwise yield that different additives may be oriented towards a single spot, which increase the risk of contamination between different additives. Moreover, the additive supplies 2, 15 preferably are oriented at an angle relative to the central axis 21 of the dispensing unit 1. Preferably, allowing the additive supplies 2, 15 to be downwardly inclined towards said central axis 21. This is beneficial since horizontally oriented additive supplies 2, 15, would result in the additive being substantially orthogonal relative to the mixing surface 18. This would result in horizontal speed remaining present after impact, which would negatively affect the laminarity of the flow of the fluid mixture. It is preferred to separate the different additives as much as possible to provide for a pure flavour of the respective additive. Since each additive supply is oriented towards its own respective mixing surface 18, there will be essentially no mixing surfaces 18 which have multiple additives thereon. Hence, contamination between flavours may be prevented as such. In case a single additive is to be added to the supply fluid, it is preferred that said additive is provided through more than one additive supply 2, 15. This may reduce the impact of the additive supply flow on the fluid mixture flow, resulting in a more controlled dispensing flow. Preferably two adjacent mixing surfaces 18 enclose an angle, but it may be conceivable that the adjacent mixing surfaces 18 comprise a mutual surface which connects the surfaces at an angle to each other. Two adjacent mixing surfaces 18 may together form a curved surface. The fluid distribution element 20 comprises both inwardly oriented fluid channels 23 and outwardly oriented fluid channels 24. The inwardly oriented mixing channels 23 are preferably inclined towards the central axis 21 of the fluid distribution element 20, whereas the outwardly oriented fluid distribution channels 24 are preferably inclined away from the central axis 21 of the fluid distribution element 20. FIG. 7 shows a cross section in a second horizontal plane of the dispensing unit, seen from an upper side of the dispensing unit 1. In this figure, for illustrative purposes parts of the dispensing unit 1 are not displayed. This figure illustrates the orientation of the duckbill valves 15, in particular the centerlines 33 thereof, with respect to the stationary mixing body 17. This figure moreover indicates the reflective upper surface 19 of the mixing body 19. The reflective surface 19 reflects light to inner parts of the dispensing unit 1 to be disinfected and moreover forms a blockage for the light between the light guidance element 11 the fluid outlet 5.

FIG. 8 shows a detailed view of a part of the dispensing unit 1 as seen from a lower side according to the invention. The figure shows the fluid distribution element 20 which is used for directing a portion of the supply fluid inwardly and a portion of the supply fluid outwardly. The fluid distribution element 20 comprises a plurality of fluid distribution channels 22. A number of said fluid distribution channels 22 are inwardly oriented fluid guiding channels 23 and a number of the fluid distribution channels 22 are outwardly oriented fluid distribution channels 24. The inwardly directed portion of the supply fluid is able to rinse the stationary mixing body 17. The outwardly directed portion of the supply fluid serves to establish a good stream for dispensing. This may be understood as a stream which does not splash excessively in all directions, but is directed in a controlled manner towards a fluid retaining element below the dispensing opening 5. It may be seen that the outwardly oriented fluid distribution channels 24 are arranged on, or debouch from, a chamfered edge 26 of the fluid distribution element 20. The inwardly oriented fluid distribution channels 23 are preferably arranged on, or debouch from, a flat or downwardly facing edge 25 of the fluid distribution element 20. FIG. 9 shows a perspective top view of a portion of the dispensing unit. In particular a portion of the supply fluid channel and distribution element is shown. The plurality of fluid guiding fins 14 are arranged circularly and divided evenly, to reduce the amount of turbulence in the supply fluid flow. Moreover, the fluid supply channel encloses, as is shown in the figure, the centrally located light guiding element 11. The fluid distribution element 20 is preferably arranged at the end of the supply fluid channel 13. The fluid channel 13 may form integral part of the distribution element 20. This means that the fluid distribution element 20 may comprise fluid guiding fins 14. In this embodiment the fluid distribution element 20 may on one side, the side facing away from the mixing chamber 3, comprise multiple fluid guiding fins 14, and on the other side, the side facing the mixing chamber 3, comprise multiple fluid distribution channels 22.

FIGS. 10a and 10b respectively show a cross section of a dispensing unit 1 according to a different embodiment in a vertical and horizontal plane. This embodiment of the disinfection element 6, in particular the UV module 10 is not centrally arranged. The UV module 10 is mounted at a side of the dispensing unit 1. A light guiding element 11 may be formed by an interior, and optionally reflective, surface of the dispensing unit 1. The light guiding element 11 may be an integral part of the wall 9 of the mixing chamber 3. Yet another difference of this embodiment is that the mixing chamber 3 is not a removable mixing module 7. However, a stationary mixing body 17 is still centrally arranged within the dispensing unit 1. Shown stationary mixing body 17 comprises a plurality of mixing surfaces 18. The backflow prevention element is in this embodiment also preferably formed by a duckbill valve 15. Said duckbill valve 15 can be predictably closed for example using a combination of a bi-directional pump such as a gear pump or, peristaltic pump, and gravity, preferably a height difference of at least 80 cm, providing the minimal negative pressure required to keep a duckbill closed once the intertia of the additive fluid is overcome by reverting the direction of the pump. Each duckbill valve 15 is directed towards a mixing surface. As can be seen in more detail in FIG. 10b, the duckbill valves 15, in particular the centerlines 33 thereof, are oriented each toward a single mixing surface 18. This way, this configuration may prevent that multiple additives are injected towards the same surface 18, which may cause a cross contamination between different additives and hence flavours. Cross-contamination may cause a dispensed fluid mixture to have a flavour which is not as requested by the user due to the undesired mixing of multiple additives. Moreover, the injected additives may highly affect the flow & laminarity of the resulting fluid mixture flow, resulting in more contamination by splashing which is undesired. Hence the mixing body 17, in particular the mixing surface 18 contribute in maintaining a more controlled fluid mixture flow. In this embodiment supply fluid is also provided into the mixing chamber 3 from the fluid supply connection 4 via a distribution element 20. Since the fluid distribution element 20 according to this embodiment is situated above the stationary mixing body 17 and hence the mixing surfaces 18, the supply fluid distributed from the fluid distribution element 20 rinses the stationary mixing body 17. This flow of supply fluid and additive along and over the mixing body 17 ensures mixing of the additive and the supply fluid into a fluid mixture. The fluid mixture may be dispensed from the fluid outlet 5.

FIG. 11 shows an embodiment of the dispensing unit 1 according to the present invention. This embodiment is not provided with a mixing body 17 as shown in the other figures, but is instead provided with a dispensing grid 35. It is however imaginable that the dispensing unit 1 is provided with both a dispensing grid 35 and a mixing body 17. The dispensing grid 35 is arranged in proximity of the discharge opening 5, which may also be referred to as the fluid outlet 5. The dispensing grid 35 comprises at least one annular ring 36, which is arranged in the mixing chamber. A plurality of spokes 38 connect an interior surface 37 of the mixing chamber with the annular ring 36. The spokes also connect the annular ring 36 with a core 40 of the dispensing grid 35. The side of the core 40 facing downwardly is cone-shaped. As can be seen, the underside of the spokes 39 is inclined downwardly. This contributes to reduce the amounts of delayed dripping by stimulating immediate drop formation and discharge at the cone-shaped core 40 at the end of a fluid dispensing session. This may moreover also reduce the phenomena where drips form on the dispensing grid 35 after the dispensing session has already been stopped. The inclined surface 39 guides any residue or mixture towards the tip of the cone. Due to the shape of the cone, less drips will reside and hence the dispensing grid 35 may stay dry after dispensing of a mixture.

The above-described inventive concepts are illustrated by several illustrative embodiments. It is conceivable that individual inventive concepts, including inventive details, may be applied without, in so doing, also applying other details of the described example. It is not necessary to elaborate on examples of all conceivable combinations of the above-described inventive concepts, as a person skilled in the art will understand numerous inventive concepts can be (re)combined in order to arrive at a specific application and/or alternative embodiment.

The ordinal numbers used in this document, like “first”, “second”, and “third” are used only for identification purposes. Hence, the use of expressions like a “second” component, does therefore not necessarily require the co-presence of a “first” component. By “complementary” components is meant that these components are configured to co-act with each other. However, to this end, these components do not necessarily have to have complementary forms. The verb “comprise” and conjugations thereof used in this patent publication are understood to mean not only “comprise”, but are also understood to mean the phrases “contain”, “substantially consist of”, “formed by” and conjugations thereof.

1. A dispending unit for dispensing mixed fluids, comprising:
at least one mixing chamber for mixing at least one supply fluid and at least one additive into a fluid mixture;
at least one fluid supply connection for providing the at least one supply fluid to the mixing chamber,
at least one additive supply for providing the at least one additive to the mixing chamber,
at least one fluid outlet for dispensing the fluid mixture from the mixing chamber,
at least one disinfection element for disinfecting at least a portion of the mixing chamber and/or the at least one additive supply.

2. The dispensing unit according to claim 1, wherein the at least one disinfection element comprises at least one UV light source.

3. The dispensing unit according to claim 2, wherein the disinfection element comprises a light guiding element, wherein the light guiding element is configured for guiding at least a part of the light emitted by the UV light source towards mixing chamber and/or additive supply.

4. The dispensing unit according to claim 1, wherein the at least one additive supply comprises at least one backflow-prevention element, and/or wherein the at least one additive supply is positioned at a distance from the at least one mixing chamber such that the at least one additive supply is not in direct contact with the at least one supply fluid and/or the fluid mixture.

5. (canceled)

6. The dispensing unit according to claim 1, comprising at least one mixing body, said mixing body at least partially accommodated in the at least one mixing chamber.

7. The dispensing unit according to claim 6, wherein the at least one additive supply is oriented in a direction towards a mixing surface of the mixing body, for allowing the at least one additive to be at least partially mixed with the at least one supply fluid in proximity of and/or on the mixing surface.

8. The dispensing unit according to claim 6, wherein the disinfection element is further configured for disinfection of at least a part of the mixing body.

9. The dispensing unit according to claim 6, wherein the mixing body is centrally positioned within the mixing chamber and wherein the mixing body comprises at least one mixing surface, and further wherein said at least one mixing surface forms part of a circumferential surface.

10. The dispensing unit according to claim 1, further comprising at least one dispensing grid, wherein the at least one dispensing grid is arranged at least partially inside the mixing chamber.

11. The dispensing unit according to claim 10, wherein the at least one dispensing grid comprises at least one annular ring and a plurality of spokes, said spokes connected to an inner surface of the mixing surface and the at least one annular ring.

12. The dispensing unit according to claim 10, further comprising at least one core, wherein a side of the core facing away from the fluid supply connection is tapered.

13. The dispensing unit according to claim 10, wherein at least a portion of the dispensing grid is lower compared to the rest of the dispensing grid.

14. The dispensing unit according to claim 10, wherein the at least one dispensing unit is at least partially arranged in direct line of sight with the at least one disinfection element.

15. The dispensing unit according to claim 1, further comprising at least one fluid distribution element, wherein the at least one fluid distribution element comprises a plurality of fluid distribution channels, wherein each channel is configured to distribute a portion of the supply fluid into the mixing chamber,
wherein the fluid distribution element comprises at least one inwardly oriented distribution channel, wherein each inwardly oriented distribution channel is inclined inwardly with respect to the central axis of the distribution element,
and/or wherein the fluid distribution element comprises at least one outwardly oriented fluid distribution channel, wherein each outwardly oriented fluid distribution channel is inclined outwardly with respect to the central axis of the distribution element.

16. (canceled)

17. (canceled)

18. (canceled)

19. The dispensing unit according to claim 1, wherein the at least one additive supply is inclined in a direction towards the mixing chamber,
and/or comprising at least one sensor, wherein said dispensing unit is configured for activating at least part of the dispensing unit upon detecting a presence of a user by the at least one sensor.

20. (canceled)

21. The dispensing unit according to claim 1, wherein the at least one fluid supply connection is configured for providing a flow of the at least one supply fluid, wherein the speed of flow of the at least one supply fluid from the at least one fluid supply connection is in the range of 1.5 L/min-4.0 L/min;
and/or wherein the at least one additive supply is configured for providing a flow of the at least one additive, wherein the speed of flow of the at least one additive from the at least one additive supply is in the range of 80-350 ml/min.
and/or wherein the at least one mixing chamber is part of a mixing module.

22. (canceled)

23. (canceled)

24. The dispensing unit according to claim 1, wherein at least one cleaning module is inserted into or insertable into the dispensing unit, wherein an inserted cleaning module is configured to define a cleaning chamber for cleaning a the supply nozzle and/or a part of the disinfection element,
wherein the cleaning module comprises at least one curved surface for redirecting the at least one supply fluid from the supply fluid connection towards elements and/or parts to be cleaned.

25. (canceled)

26. The dispensing unit according to claim 1, further comprising at least one control unit, said control unit configured for controlling of a predetermined mixing ratio of the amount of supply fluid and the amount of at least one additive,
wherein the control unit is configured to stop the supply of additive prior to stopping the supply of fluid supply, wherein the predetermined mixing ratio is maintained.

27. (canceled)

28. (canceled)

29. A method for dispensing flavoured water, comprising a dispensing unit, said dispensing unit comprising a mixing body with at least one mixing surface, comprising the steps of:
a) providing at least one water stream at least partially onto a mixing surface;
b) providing at least one additive by an additive supply, wherein each additive supply is positioned opposite of a mixing surface and the additive is provided onto the opposite mixing surface;
c) decreasing the speed of a part of the water stream of step a) and/or the additive supply of step b),
wherein step b) is ended prior to ending step a), rinsing additive residue on the mixing body.

30. The method according to claim 29, wherein step c) is achieved by letting the part of the water stream and/or additive supply hit a mixing surface of the mixing body at least a different part of a mixing surface, of the mixing body.
